(19) United States
(12) Patent Application Publication
(10) Pub. No.: US 2008/0090812 A1
(43) Pub. Date: Apr. 17, 2008

(54) Pyrrolobenzodiazepine Therapeutic Agents Useful in the Treatment of Leukemias

(75) Inventors: Christopher John Pepper, Vale of Glamorgan (GB); David Edwid Thurston, London (GB)

Correspondence Address: MICHAEL BEST & FRIEDRICH LLP, ONE SOUTH PINCKNEY STREET, P O BOX 1806, MADISON, WI, 53701, US

(73) Assignee: SPIROGEN LIMITED, Ryde, Isle of Wight (GB)

(21) Appl. No.: 11/569,007

(22) PCT Filed: May 5, 2005
(86) PCT No.: PCT/GB05/01881

§ 371 Date: Nov. 13, 2006

(30) Foreign Application Priority Data
May 13, 2004 (GB) 0410725.6

(51) Int. Cl. A61K 31/5517; A61P 35/02

(52) U.S. Cl. 514/220

(57) ABSTRACT

A pyrrolobenzodiazepine dimer compound of Formula (I): or pharmaceutically acceptable salt or solvate thereof is useful as a therapeutic agent for the treatment of leukaemias, especially B-cell leukaemias, that exhibit resistance to other chemotherapeutic drugs, wherein: the dotted lines indicate the optional presence of a double bond between C1 and C2 or C2 and C3; R² and R³ are independently selected from —H, ═O, ═CH₂, —CN, —R, OR, halo, ═CH—R, O—SO₂—R, CO₂R and COR; R⁶, R⁷ and R⁹ are independently selected from II, R, OII, OR, SII, SR, NII₂, NIIR, NRR′, nitro, Me₃Sn and halo; where R and R′ are independently selected from optionally substituted C₁₋₁₂ alkyl, C₃₋₂₀ heterocyclyl and C₅₋₂₀ aryl groups; R¹⁰ is a carbamate-based nitrogen protecting group and R¹⁵ is either O—R¹¹, wherein R is an oxygen protecting group, or OH, or R¹⁰ and R¹⁵ together form a double bond between N10 and C11; R″ is a C₃₋₁₂ alkylene group, which chain may be interrupted by one or more heteroatoms and/or aromatic rings, and each X is independently selected from 0, S, or NH; R^{2′}, R^{3′}, R^{6′}, R^{7′}, R^{9′}, R^{10′} and R^{15′} are all independently selected from the same lists as previously defined for R², R³, R⁶, R⁷, R⁹, R¹⁰ and R¹⁵ respectively.

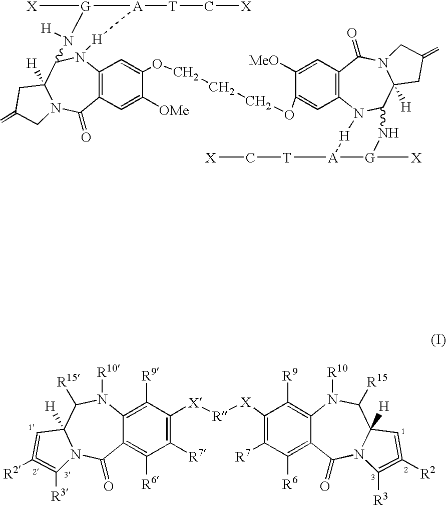

The present invention relates to pyrrolobenzodiazepine (PBD) dimer therapeutic agents useful in the treatment of leukaemias, especially B-cell leukaemias, that exhibit a resistance to other chemotherapeutic drugs.

### BACKGROUND

The development of drug resistance is one of the most important problems encountered in cancer chemotherapy as up to 50% of patients' cancers have de novo drug resistance or develop resistance to anticancer drugs.

Studies indicate that most, if not all, chemotherapeutic agents exert their anticancer activity by inducing apoptosis; therefore resistance to apoptosis may be a major factor limiting the effectiveness of anticancer therapy (Curr. Med. Chem. Anti-Canc. Agents, 2002, 2(3), 387-401). Much research has been conducted into apoptosis as an active mechanism of cell death (Wien. Klin. Wochenschr., 2003, 115(15-16), 563-574) and into the clinical use of DNA damaging agents to activate tumour suppression responses triggered by DNA damage to induce apoptosis in damaged cells (Apoptosis, 2000, 5(6), 491-507).

Resistance to the cytotoxic effects of chemotherapeutic agents has been observed in a number of different human cancers. Drug resistance has manifested itself in human leukaemias as both single drug resistance (Oncogene, 2003, 22(47), 7389-7395; Curr. Opin. Hematol., 2002, 9(4), 303-307; Leukaemia, 2003, 17(9), 1794-1805) and multidrug resistance (Int. J. Hematol., 2000, 72(3), 290-297). Many different mechanisms have been proposed for how cancers, and especially liquid malignancies, develop drug resistance (Vet. Clin. North Am. Small Anim. Pract., 2003, 33(3), 651-667).

Tumour suppression responses are thought to be regulated, at least in part, by the p53 protein. This may act either to initiate DNA repair mechanisms or to activate mechanisms that lead to apoptotic destruction of the cell (Cancer Lett., 1998, 131(1), 85-99). However some tumours are p53 mutant or p53 null hence reducing or even eliminating the p53 mediated pathway as a possible mechanism for control of apoptosis.

It is known that some of the chemotherapeutics used in the treatment of B-cell leukaemias deplete immunological T-cells as well as killing malignant B-cells. This has been shown for fludarabine (Blood, 1998, 91(5), 1742-1748; Br. J. Hematol., 1995, 91(2), 341-344) and for chlorambucil (Cell Cycle, 2003, 2(1), 53-58).

Many chemotherapeutics also show significant cytotoxicity towards non-malignant cells as well as inducing apoptosis in malignant cells.

Pyrrolobenzodiazepines (PBDs) are known in the art, some of which have the ability to recognise and bond to specific sequences of DNA. PBDs are of the general structure:

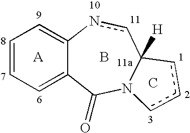

They differ in the number, type and position of substituents, in both their aromatic A rings and pyrrolo C rings, and in the degree of saturation of the C ring. In the B-ring there is either an imine (N═C), a carbinolamine(NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). Their ability to form an adduct in the minor groove, enables them to interfere with DNA processing, hence their use as antitumour agents.

### SUMMARY OF THE INVENTION

The present invention provides a group of cytotoxic agents that remain active against some drug resistant leukaemias and that appear to operate via a p53 independent pathway. Such chemotherapeutic agents present useful treatments for drug resistant or p53 mutant or p53 null leukaemias.

The present invention also provides a group of cytotoxic agents that induce apoptosis preferentially in B-cells over T-cells in a mixture of B- and T- cells either in vitro or in vivo from a patient with B-cell leukaemia.

In addition, the present invention provides a group of cytotoxic agents that induce apoptosis preferentially in malignant cells over non-malignant cells in a mixture of malignant and non-malignant cells either in vitro or in vivo from a patient with B-cell leukaemia.

In a first aspect, the present invention relates to the treatment of a patient suffering from leukaemia that exhibits drug resistance, comprising administering to said patient a therapeutically effective amount of a compound of formula I:

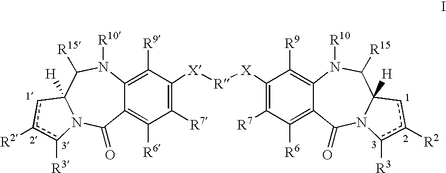

or pharmaceutically acceptable salt or solvate thereof, wherein: the dotted lines indicate the optional presence of a double bond between C1 and C2 or C2 and C3;

R² and R³ are independently selected from —H, ═O, ═CH₂, —CN, —R, OR, halo, ═CH—R, O—SO₂—R, CO₂R and COR;

R⁶, R⁷ and R⁹ are independently selected from H, R, OH, OR, SH, SR, NH₂, NHR, NRR′, nitro, Me₃Sn and halo;

where R and R′ are independently selected from optionally substituted C₁₋₁₂ alkyl, C₃₋₂₀ heterocyclyl and C₅₋₂₀ aryl groups;

R¹⁰ is a carbamate-based nitrogen protecting group and R¹⁵ is either O—R¹¹, wherein R¹¹ is an oxygen protecting group, or OH, or R¹⁰ and R¹⁵ together form a double bond between N10 and C11;

R″ is a C₃₋₁₂ alkylene group, which chain may be interrupted by one or more heteroatoms, e.g. O, S, NH, and/or aromatic rings, e.g. benzene or pyridine, and each X is independently selected from O, S, or NH;

R^{2′}, R^{3′}, R^{6′}, R^{7′}, R^{9′}, R^{10′} and R^{15′} are all independently selected from the same lists as previously defined for R², R³, R⁶, R⁷, R⁹, R¹⁰ and R¹⁵ respectively.

Preferably the leukaemia is a B-cell chronic lymphocytic leukaemia (B-CLL)

The molecules of formula I are known to interact in the minor groove of DNA to form a cross link between bases located on opposite strands of the DNA. In some cases, it is believed that the molecular structure of the compound of formula I allows hydrogen bonding interactions between the compound and certain molecular features of the DNA bases as shown in FIG. 1 for a preferred compound of formula I.

Since compounds of formula I are DNA cross-linking agents, it may be expected that p53-mediated detection of these adducts would result in up-regulation of nucleotide excision repair mechanisms. However, in some cases the binding of compounds of formula I to DNA does not elicit these responses in leukaemias. In fact, compounds of formula I may remain active against leukaemias with p53 mutations which eliminate p53-mediated detection of the DNA cross-linked adducts.

In a second aspect, the present invention relates to the treatment of a patient suffering from B-cell leukaemia wherein it is desired not to reduce the patient's T-cell count, comprising administering to said patient a therapeutically effective amount of a compound of formula I, or pharmaceutically acceptable salt or solvate thereof.

One of the undesirable aspects of many current B-cell leukaemia treatments is that the chemotherapeutic drugs used exhibit a similar cytotoxicity towards both T-cells and B-cells. This has the result that even though the malignant B-cells are killed by the drug, the patient's immune system is also considerably weakened by killing of the T-cells in similar numbers. This may have the result that the patient becomes more vulnerable to secondary infection.

Compounds according to the present invention and pharmaceutical preparations thereof preferably exhibit a higher cytotoxicity, i.e. lower LD₅₀, for B-cells than for T-cells, in cells from healthy patients and in cells from those suffering from B-CLL.

When compared to existing chemotherapeutic agents, the difference between the LD₅₀ values for T-cells and for B-cells, when considered as a percentage of the LD₅₀ in B-cells, is a positive value and is preferably larger for compounds of formula I than for existing chemotherapeutic agents. In other words, the formula A below has a positive value and is preferably larger for compounds of formula I than for existing chemotherapeutic agents.

A═[(LD₅₀(T cells)−LD₅₀(B cells))/LD₅₀(B cells)]×100

Thus the compounds of formula I preferably show a greater selective killing of leukaemic B-cells over T-cells than existing B-CLL therapeutic agents.

Preferably this positive value difference between the LD₅₀ values for T-cells and for B-cells, when considered as a percentage of the LD₅₀ in B-cells, for compounds or pharmaceutically acceptable compositions of the present invention is in the ratio of at least 1.2:1, preferably at least 1.5:1, preferably at least 2:1, more preferably at least 5:1 and most preferably at least 10:1, with the value for existing chemotherapeutic agents. In other words, the values of formula A above are in the following ratios:

A(compounds of the present invention):A(existing chemotherapeutic agents)═at least 1.2:1, preferably at least 1.5:1, preferably at least 2:1, more preferably at least 5:1 and most preferably at least 10:1

In a third aspect, the present invention relates to the treatment of a patient suffering from B-cell leukaemia wherein it is desired to selectively kill malignant B-cells, comprising administering to said patient a therapeutically effective amount of a compound of formula I, or pharmaceutically acceptable salt or solvate thereof.

An important aim in terms of drug development for the treatment of cancer is to produce a compound that selectively kills cancer cells without inducing significant cytotoxicity in the surrounding normal tissue. Unfortunately, no differential tumour-selective therapeutic index exists for most of the currently used drugs and substantial normal tissue damage is encountered during cancer therapy. This can be one of the most significant limiting factors in determining the dose and schedule of a new agent. Naturally, individual cell types have different susceptibilities to anti-cancer drugs and this is dependant on a number of factors including the proliferative index of the cells and their innate tendency to succumb to or resist cell death signals.

Preferably compounds and pharmaceutically acceptable compositions of the present invention show a higher cytotoxicity towards malignant B-CLL cells than towards normal B-cells. More preferably the ratio of the LD₅₀ for compounds and pharmaceutically acceptable compositions of the present invention in B-CLL cells and the LD₅₀ in normal B-cells is at least 2:1, preferably at least 5:1, more preferably at least 10:1.

Furthermore, the present invention relates to the use of PBD dimers of formula I in the manufacture of a medicament for the treatment of leukaemias that exhibit drug resistance.

In another aspect, the present invention relates to the use of PBD dimers of formula I in the manufacture of a medicament for the treatment of B-cell leukaemias, wherein it is desired not to reduce the patient's T-cell count.

The present invention also relates to the use of PBD dimers of formula I in the manufacture of a medicament useful for the treatment of B-cell leukaemias wherein it is desired to selectively kill malignant B-cells.

In other words, the compounds of formula I are useful in the manufacture of chemotherapeutic agents for the treatment of B-cell leukaemias wherein it is not desired to reduce a patient's T-cell count or wherein it is desired to selectively kill malignant B-cells.

### DESCRIPTION OF THE FIGURES

FIG. 1 illustrates a possible binding mode for a compound of formula I to DNA.

FIG. 2 shows a comparison of the in vitro sensitivity to a compound of formula I (SJG-136) of samples taken from 34 B-CLL patients. FIG. 2a shows cytotoxicity compared using LD₅₀ values (±SD). FIG. 2b shows LD₉₀ values (±SD) derived from in vitro cultures of B-CLL cells exposed to SJG-136 (10⁻¹−10⁻⁷ M) for 48 h. Results represent the means (±SD) of three independent experiments.

FIG. 3 illustrates a comparison of the cytotoxic effect of a compound of formula I (SJG-136) on treated versus untreated and V_{H} gene mutated versus unmutated B-CLL cells. FIG. 3a shows the mean LD₅₀ values calculated from the dose-response curves derived from a flow cytometric apoptosis assay indicating drug sensitivity between previously treated and untreated B-CLL cells. FIG. 3b shows the cytotoxicity of SJG-136 in B-CLL samples derived from patients with mutated or unmutated immunoglobulin V_{H} genes.

FIG. 4 shows the activation of p53 and induction of GADD45 expression in cells treated with the cross-linking agents chlorambucil and a compound of formula I (SJG-136). FIG. 4a shows results for p53 activation in B-CLL cells cultured for 4 h in the presence of chlorambucil or SJG-136, or in the absence of drug as a control. FIG. 4b shows induction of GADD45 expression in B-CLL cells in response to chlorambucil and SJG-136 as quantified by flow cytometry in units of mean fluorescence intensity.

FIG. 5 shows a comparison of the cytotoxicity of a compound of formula I (SJG-136) and fludarabine in samples derived from previously treated and untreated B-CLL patients.

FIG. 6 shows a comparison of the cytotoxicity of a compound of formula I (SJG-136) in B-CLL cells and normal lymphocytes. Cytotoxicity was compared using LD₅₀ values (±SD) derived from in vitro cultures of malignant B-cells and T-cells derived from B-CLL samples and the B- and T-lymphocyte sub-sets from normal age-matched control samples.

FIGS. 7a and 7b show comparisons of the cytotoxicity of chlorambucil and fludarabine respectively in B-CLL cells and normal lymphocytes. Cytotoxicity was compared using LD₅₀ values (±SD) derived from in vitro cultures of malignant B-cells and T-cells derived from B-CLL samples and the B- and T-lymphocyte sub-sets from normal age-matched control samples.

FIG. 8 shows a comparison of the in vitro sensitivity to a compound of formula I (SJG-136) of samples taken from 46 B-CLL patients. FIG. 8a shows cytotoxicity compared using LD₅₀ values (±SD). FIG. 8b shows LD₉₀ values (±SD) derived from in vitro cultures of B-CLL cells exposed to SJG-136 (10⁻¹⁰−10⁻⁷ M) for 48 . Results represent the means (±SD) of three independent experiments.

FIG. 9 illustrates a comparison of the cytotoxic effect of a compound of formula I (SJG-136) on treated versus untreated and V_{H} gene mutated versus unmutated B-CLL cells. FIG. 9a shows the mean LD₅₀ values calculated from the dose-response curves derived from a flow cytometric apoptosis assay indicating drug sensitivity between previously treated and untreated B-CLL cells. FIG. 9b shows the cytotoxicity of SJG-136 in B-CLL samples derived from patients with mutated or unmutated immunoglobulin V_{H} genes.

FIG. 10 shows a comparison of the cytotoxicity of a compound of formula I (SJG-136) and fludarabine in samples derived from previously treated and untreated B-CLL patients.

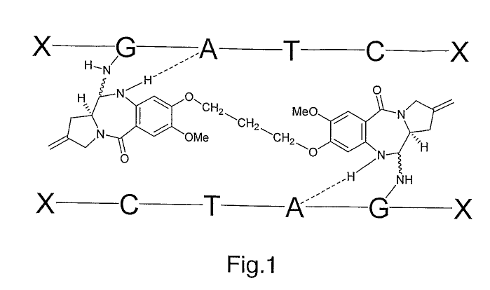

### DEFINITIONS

### Carbamate-based Nitrogen Protecting Groups

Carbamate-based nitrogen protecting groups are well known in the art, and have the following structure:

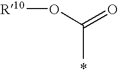

wherein R′¹⁰ is R as defined above. A large number of suitable groups are described on pages 503 to 549 of Greene, T. W. and Wuts, G. M., Protective Groups in Organic Synthesis, 3^{rd} Edition, John Wiley & Sons, Inc., 1999, which is incorporated herein by reference.

Particularly preferred protecting groups include Alloc, Troc, Fmoc, CBz, Teoc, BOC, Doc, Hoc, TCBOC, 1-Adoc and 2-Adoc.

Also suitable for use in the present invention are nitrogen protecting group which can be removed in vivo (e.g. enzymatically, using light) as described in WO 00/12507, which is incorporated herein by reference. Examples of these protecting groups include:

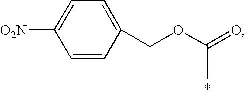

which is nitroreductase labile (e.g. using ADEPT/GDEPT);

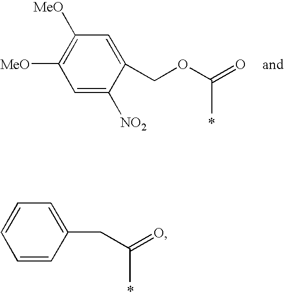

which are photolabile; and

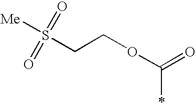

which is glutathione labile (e.g. using NPEPT).

### Oxygen Protecting Groups

Oxygen protecting groups are well known in the art. A large number of suitable groups are described on pages 23 to 200 of Greene, T. W. and Wuts, G. M., Protective Groups in Organic Synthesis, 3^{rd} Edition, John Wiley & Sons, Inc., 1999, which is incorporated herein by reference.

Classes of particular interest include silyl ethers, methyl ethers, alkyl ethers, benzyl ethers, esters, benzoates, carbonates, and sulfonates.

Preferred oxygen protecting groups include TBS, THP for the C11 oxygen atom.

It may also be preferred that any protecting groups used during the synthesis and use of compounds of formula I are orthogonal to one another. However, it is often not necessary, but may be desirable, for the carbamate-based nitrogen protecting group and R¹¹ to be orthogonal to one another, depending on whether the compound of formula I is to be used with the nitrogen protecting group in place.

### Substituents

The phrase “optionally substituted” as used herein, pertains to a parent group which may be unsubstituted or which may be substituted.

Unless otherwise specified, the term “substituted” as used herein, pertains to a parent group which bears one or more substitutents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known.

Examples of substituents are described in more detail below.

C₁₋₁₂ alkyl: The term “C₁₋₁₂ alkyl” as used herein, pertains to a monovalent moiety obtained by removing a hydrogen atom from a carbon atom of a hydrocarbon compound having from 1 to 12 carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂) , propyl (C₃), butyl (C₄) , pentyl (C₅) , hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇).

Examples of saturated branched alkyl groups include iso-propyl (C₃) , iso-butyl (C₄), sec-butyl (C₄), tert-butyl (C₄), iso-pentyl (C₅), and neo-pentyl (C₅).

C₂₋₁₂ Alkenyl: The term “C₂₋₁₂ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₁₂ alkynyl: The term “C₂₋₁₂ alkynyl” as used herein, pertains to an alkyl group having one or more carbon-carbon triple bonds.

Examples of unsaturated alkynyl groups include, but are not limited to, ethynyl (ethinyl, —C═CH) and 2-propynyl (propargyl, —CH₂—C═CH)

C₃₋₁₂ cycloalkyl: The term “C₃₋₁₂ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:

saturated monocyclic hydrocarbon compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);

unsaturated monocyclic hydrocarbon compounds: cyclopropene (C₃), cyclobutene (C₄), cyclopentene (C₅), cyclohexene (C₆), methylcyclopropene (C₄), dimethylcyclopropene (C₅), methylcyclobutene (C₅) , dimethylcyclobutene (C₆), methylcyclopentene (C₆), dimethylcyclopentene (C₇) and methylcyclohexene (C₇); and

saturated polycyclic hydrocarbon compounds: norcarane (C₇) , norpinane (C₇) , norbornane (C₇).

C₃₋₂₀ heterocyclyl: The term “C₃₋₂₀ heterocyclyl” as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆ heterocyclyl”, as used herein, pertains to a heterocyclyl group having 5 or 6 ring atoms.

Examples of monocyclic heterocyclyl groups include, but are not limited to, those derived from: N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁Oₗ: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

C₅₋₂₀ aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. Preferably, each ring has from 5 to 7 ring atoms.

In this context, the prefixes (e.g. C₃₋₂₀, C₅₋₇, C₅₋₆, etc.) denote the number of ring atoms, or range of number of ring atoms, whether carbon atoms or heteroatoms. For example, the term “C₅₋₆ aryl” as used herein, pertains to an aryl group having 5 or 6 ring atoms.

The ring atoms may be all carbon atoms, as in “carboaryl groups”. Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅) , isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Examples of heteroaryl which comprise fused rings, include, but are not limited to:

C₉ (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);

C₁₀ (with 2 fused rings) derived from chromene (O₁), isochromene (O₁), chroman (O₁), isochroman (O¹), benzodioxan (O₂) quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);

C₁₁ (with 2 fused rings) derived from benzodiazepine (N₂);

C₁₃ (with 3 fused rings) derived from carbazole (N₁), dibenzofuran (O₁), dibenzothiophene (S₁), carboline (N₂), perimidine (N₂), pyridoindole (N₂); and,

C₁₄ (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₂S₁), thianthrene (S₂), phenanthridine (N₁), phenanthroline (N₂), phenazine (N₂).

The above groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Alkoxy: —OR, wherein R is an alkyl group, for example, a C₁₋₇ alkyl group. Examples of C₁₋₇ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR¹)(OR²), wherein R¹ and R² are independently acetal substituents, for example, a C₁₋₇ alkyl group, a C₃₋₂₀ heterocyclyl group, or a C₅₋₂₀ aryl group, preferably a C₁₋₇ alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe) (OEt).

Hemiacetal: —CH(OH) (OR¹), wherein R¹ is a hemiacetal substituent, for example, a C₁₋₇ alkyl group, a C₃₋₂₀ heterocyclyl group, or a C₅₋₂₀ aryl group, preferably a Cₗ₋₇ alkyl group. Examples of hemiacetal groups include, but are not limited to, —CH(OH)(OMe) and —CH(OH)(OEt).

Ketal: —CR(OR¹) (OR²) , where R¹ and R² are as defined for acetals, and R is a ketal substituent other than hydrogen, for example, a C₁₋₇ alkyl group, a C₃₋₂₀ heterocyclyl group, or a C₅₋₂₀ aryl group, preferably a C₁₋₇ alkyl group. Examples ketal groups include, but are not limited to, —C(Me) (OMe)₂, —C(Me) (OEt)₂, —C (Me) (OMe) (OEt), —C(Et) (OMe)₂, —C(Et) (OEt)₂, and —C(Et) (OMe) (OEt).

Hemiketal: —CR(OH) (OR¹), where R¹ is as defined for hemiacetals, and R is a hemiketal substituent other than hydrogen, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —C(Me)(OH) (OMe), —C(Et)(OH)(OMe), —C(Me)(OH)(OEt), and —C(Et)(OH)(OEt).

Oxo (keto, —one): ═O.

Thione (thioketone): ═S.

Imino (imine): ═NR, wherein R is an imino substituent, for example, hydrogen, C₁₋₇ alkyl group, a C₃₋₂₀ heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —OC(═O)OCH₃, —OC(═O)OCH₂CH₃, —OC(═O)OC(CH₃)₃, and —OC(═O)OPh.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R² is an acyl substituent, for example, a C₁₋₇ alkyl group, a C₃₋₂₀ heterocyclyl group, or a C₅₋₂₀ aryl group, preferably hydrogen or a C₁₋₇ alkyl group. Examples of acylamide groups include, but are not limited to, —NHC(═O)CH₃ , —NHC(═O)CH₂CH₃, and —NHC(═O)Ph. R¹ and R² may together form a cyclic structure, as in, for example, succinimidyl, maleimidyl, and phthalimidyl:

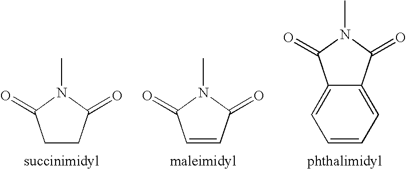

Aminocarbonyloxy: —OC(═O)NR¹R², wherein R¹ and R² are independently amino substituents, as defined for amino groups. Examples of aminocarbonyloxy groups include, but are not limited to, —OC(═O)NH₂, —OC(═O)NHMe, —OC (═O)NMe₂, and —OC(═O)NEt₂.

Ureido: —N R¹) CONR²R³ wherein R² and R³ are independently amino substituents, as defined for amino groups, and R¹ is a ureido substituent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂.

NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

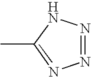

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇ alkyl group. Examples of imino groups include, but are not limited to, ═NH, ═NMe, and ═NEt.

Amidine (amidino) : —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇ alkyl group. Examples of amidine groups include, but are not limited to, —C(═NH)NH₂, —C(═NH)NMe₂, and —C(═NMe)NMe₂.

Nitro: —NO₂.

Nitroso: —NO.

Azido: —N₃.

Cyano (nitrile, carbonitrile): —CN.

Isocyano: ——NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfine (sulfinyl, sulfoxide) : —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfone (sulfonyl) : —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group, including, for example, a fluorinated or perfluorinated C₁₋₇alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl) , —S (═O)₂CH₂CF₃ (tresyl), —S (═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇ alkyl group, a C₃₋₂₀ heterocyclyl group, or a C₅₋₂₀ aryl group, preferably a C₁₋₇ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇ alkyl group, a C₃₋₂₀ heterocyclyl group, or a C₅₋₂₀ aryl group, preferably a C₁₋₇ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₃₋₇ alkyl group. Examples of sulfinyloxy groups include, but are not limited to, —OS(═O)CH₃ and —OS(═O) CH₂CH₃.

Sulfonyloxy: —OS(═O)₂R, wherein R is a sulfonyloxy substituent, for example, a C₁₋₇ alkyl group, a C₃₋₂₀heterocyclyl group, or a C₅₋₂₀ aryl group, preferably a C₁₋₇ alkyl group. Examples of sulfonyloxy groups include, but are not limited to, —OS(═O)₂CH₃ (mesylate) and —OS(═O)₂CH₂CH₃ (esylate). sulfate: —OS(═O)₂OR; wherein R is a sulfate substituent, for example, a C₁₋₇ alkyl group, a C₃₋₂₀ heterocyclyl group, or a C₅₋₂₀ aryl group, preferably a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N (CH₃)S(═O)₂C₆H₅.

Phosphino (phosphine) : —PR₂, wherein R is a phosphino substituent, for example, —H, a C₁₋₇ alkyl group, a C₃₋₂₀ heterocyclyl group, or a C₅₋₂₀ aryl group, preferably —H, a Cₗ₋₇ alkyl group, or a C₅₋₂₀ aryl group. Examples of phosphino groups include, but are not limited to, —PH₂, —P(CH₃)₂, —P(CH₂CH₃)₂, —P(t—Bu)₂, and —P(Ph)₂.

Phospho: —P(═O)₂.

Phosphinyl (phosphine oxide): —P(═O)R₂, wherein R is a phosphinyl substituent, for example, a C₁₋₇ alkyl group, a C₃₋₂₀ heterocyclyl group, or a C₅₋₂₀ aryl group, preferably a C₁₋₇ alkyl group or a C₅₋₂₀ aryl group. Examples of phosphinyl groups include, but are not limited to, —P(═O) (CH₃)2, —P(═O) (CH₂CH₃)₂, —P(═O) (t—Bu)₂, and —-P(═O) (Ph)₂.

Phosphonic acid (phosphono) : —P(═O) (OH)₂.

Phosphonate (phosphono ester) : —P(═O) (OR)₂, where R is a phosphonate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphonate groups include, but are not limited to, —P(═O) (OCH₃)₂, —P(═O) (OCH₂CH₃)₂, —P(═O) (O—t—Bu)₂, and —P(═O) (OPh)₂.

Phosphoric acid (phosphonooxy): —OP (═O) (OH)₂.

Phosphate (phosphonooxy ester): —OP(═O) (OR)₂, where R is a phosphate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphate groups include, but are not limited to, —OP(═O) (OCH₃)₂, —OP(═O) (OCH₂CH₃)₂, —OP(═O) (O—t—Bu)₂, and —OP(═O) (OPh)₂.

Phosphorous acid: —OP(OH)₂.

Phosphite: —OP(OR)₂, where R is a phosphite substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphite groups include, but are not limited to, —OP(OCH₃)₂, —OP(OCH₂CH₃)₂, —OP(O—t—Bu)₂, and —OP(OPh)₂.

Phosphoramidite: —OP(OR¹)—NR²₂, where R¹ and R² are phosphoramidite substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i—Pr)₂, and —OP(OCH₂CH₂CN)—N(i—Pr)₂.

Phosphoramidate: —OP(═O) (OR¹)—NR²₂, where R¹ and R² are phosphoramidate substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O) (OCH₂CH₃)—N(CH₃)₂, —OP(═O) (OCH₂CH₃)—N (i—Pr)₂, and —OP(═O) (OCH₂CH₂CN)—N(i—Pr)₂.

### Alkylene

c₃₋₁₂ alkylene: The term “C₃₋₁₂ alkylene”, as used herein, pertains to a bidentate moiety obtained by removing two hydrogen atoms, either both from the same carbon atom, or one from each of two different carbon atoms, of a hydrocarbon compound having from 3 to 12 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Examples of linear saturated C₃₋₁₂ alkylene groups include, but are not limited to, —(CH₂)ₙ— where n is an integer from 3 to 12, for example, —CH₂CH₂CH₂— (propylene), —CH₂CH₂CH₂CH₂— (butylene), —CH₂CH₂CH₂CH₂CH₂— (pentylene) and —CH₂CH₂CH₂CH₂CH₂CH₂CH₂— (heptylene)

Examples of branched saturated C₃₋₁₂ alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH (CH₂CH₃)CH₂—, and —CH₂CH (CH₂CH₃)CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated C₃₋₁₂ alkylene groups (C₃₋₁₂ cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

### Drug-resistance

As used herein, the term “drug resistance” refers to a property displayed by cancers that have been treated for a first time with a specific chemotherapeutic agent, and when treated for a subsequent time with the same chemotherapeutic agent, i.e. for a second or further time, show an LD₅₀ higher than the one observed in the first treatment i.e the cancer has developed a resistance to that particular chemotherapeutic agent.

Furthermore, “drug resistance” as used herein may refer to a property displayed by cancers that have been treated for a first time with a specific chemotherapeutic agent and when the cancer is treated for a subsequent time, i.e. for a second or further time, with a second chemotherapeutic agent, the LD₅₀ of the second agent is raised compared to the expected LD₅₀ for the second agent, where the expected LD₅₀ for the second agent is the LD₅₀ for treating the same cancer which has not been previously treated, i.e. the cancer has developed a resistance to the second chemotherapeutic agent in spite of not having been previously treated with the second agent.

In some cases, cancers may exhibit a resistance to certain chemotherapeutic agents or classes of chemotherapeutic agents from the outset, i.e. the resistance is inherent in the cancer and is exhibited the first time the drug is administered. In the majority of cases of drug resistance however, resistance builds up due to repeated administration of one or more chemotherapeutic agents.

There are many different biochemical mechanisms by which tumour cells develop resistance to chemotherapeutic drugs, these include:

1. Decreased intracellular drug levels. This could result from increased drug efflux or decreased inward transport. Among the drugs which become resistant by this mechanism are the anthracyclines, dactinomycin, vinca alkaloids, and epipodophyllotoxins;

2. Increased drug inactivation. Included in this group are the alkylating agents, antimetabolites and bleomycin;

3. Deceased conversion of drug to an active form. This mechanism is most common among the antimetabolites which must be converted to the nucleotide before they are active;

4. Altered amount of target enzyme or receptor (gene amplification). Methotrexate is a classic example here as often in methotrexate resistant tumors there is an amplification in the target enzyme dihydrofolate reductase;

5. Decreased affinity of target enzyme or receptor for drug. Examples, here are the antimetabolites and hydroxyurea;

6. Enhanced repair of the drug-induced defect. The alkylating agents typically show resistance by this mechanism although other mechanisms are also important with these drugs;

7. Decreased activity of an enzyme required for the killing effect (e.g. topoisomerase II). Decreased activity of this enzyme is important for resistance to doxorubicin, m-AMSA, and the epipodophyllotoxins;

8. Multidrug Resistance (MDR) This is a phenomenon whereby tumors become resistant to several, often unrelated drugs, simultaneously. The multidrug resistance (MDR1) gene encodes an ATP-dependent efflux pump, called p-glycoprotein, that may become amplified in drug-resistant tumours. MDR activity may be reversed by drugs such as calcium channel blockers (e.g., verapamil), cyclosporin, or tamoxifen. Multidrug resistance occurs between several different structurally unrelated anti-tumour agents that apparently have different mechanisms of action. This resistance is obtained through stepwise selection and it reflects the amplification of a gene that encodes a transmembrane protein that pumps the drugs out of the cell. Thus the resistant cell maintains a lower intracellular drug level than the drug-sensitive parental cells. The degree of P-glycoprotein overproduction has been correlated with the degree of drug resistance in a number of human cancers.

In addition to the examples given above of the “induction” of resistance it should be noted that “selection” also plays a key role in the development of drug resistance. Within all tumour populations there is intrinsic variation in gene, and hence protein, expression levels. Once the tumour is exposed to chemotherapy the sub-clones with the most resistant genotype/phenotype are effectively selected because the more sensitive sub-clones are killed. The surviving tumour cells go on to divide and repopulate the tumour mass and subsequent rounds of therapy result in a diminished response because of this drug-induced skewing of the tumour population. In most human malignancies the development of drug resistance is likely to be the result of a complex mixture of induction and selection. Of the mechanisms listed above for increased drug resistance, numbers 2 and 6 are the most relevant for increased resistance to known PDB cross-linking agents.

The compounds of the present invention may be useful in the treatment of both leukaemias that exhibit drug resistance from the outset and those that exhibit drug resistance in response to treatment with chemotherapeutic agents.

### Methods of Treatment

As described above, the present invention provides the use of a compound of formula I, or a pharmaceutically acceptable salt or solvate thereof, in a method of therapy. It is preferred that the compound of formula I is administered in the form of a pharmaceutical composition.

The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of medical doctors.

A compound may be administered alone or in combination with other treatments, either simultaneously or sequentially dependent upon the condition to be treated. Examples of treatments and therapies include, but are not limited to, chemotherapy (the administration of active agents, including, e.g. drugs); surgery; and radiation therapy. If the compound of formula I bears a carbamate-based nitrogen protecting group which may be removed in vivo, then the methods of treatment described in WO 00/12507 (ADEPT, GDEPT and PDT) may be used.

Pharmaceutical compositions according to the present invention, and for use in accordance with the present invention, may comprise, in addition to the active ingredient, i.e. a compound of formula I, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

In pharmaceutical compositions of the present invention which comprise a compound of formula I and a solvent, the compound of formula I may preferably be present in its carbinolamine or carbinolamine ether form.

### Includes Other Forms

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻0), a salt or solvate thereof, as well as conventional protected forms.

### Isomers, Salts and Solvates

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r- forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal- forms; α- and β-forms; axial and equatorial forms; boat-, chair-, twist-, envelope-, and halfchair-forms; and combinations thereof, hereinafter collectively referred to as “isomers” (or “isomeric forms”).

In some embodiments, compounds of the present invention have the following stereochemistry at the C11 position:

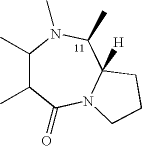

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

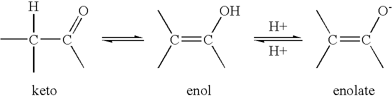

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g. —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e. NH₄+) and substituted ammonium ions (e.g. NH₃R+, NH₂R₂+, NHR₃+, NR₄+). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄+.

If the compound is cationic, or has a functional group which may be cationic (e.g. —NH₂ may be —NH₃+), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri- hydrate, etc.

Solvates of particular relevance to the present invention are those where the solvent adds across the imine bond of the PBD moiety, which is illustrated below where the solvent is water or an alcohol (R^{B}OH, where R^{B} is an ether substituent as described above e.g. MeOH):

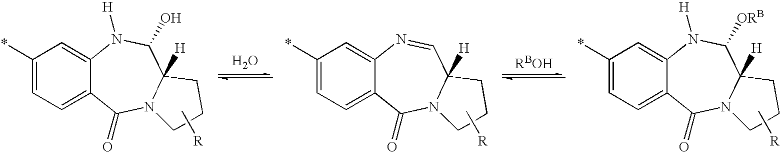

wherein * represents the dimer link to the other PBD moiety. These forms can be called the carbinolamine and carbinolamine ether forms of the PBD. The balance of these equilibria depend on the conditions in which the compounds are found, as well as the nature of the moiety itself.

In general any nucleophilic solvent is capable of forming such solvates as illustrated above for hydoxylic solvents. Other nucleophilic solvents include thiols and amines.

These solvates may be isolated in solid form, for example, by lyophilisation.

### General Synthetic Methods

The synthesis of PBD compounds is extensively discussed in WO 00/12508, which discussion is incorporated herein by reference.

Alternative methods of synthesising N10 protected PBDs are disclosed in co-pending application GB 0321295.8 (filed 11 Sep. 2003), which describes the use of isocyanate intermediates.

The synthesis of compounds of formula I in WO/12508 was achieved by formation of the dimeric backbone comprising the assembled A and C rings linked through the A ring by the diether linking chain. The N10 position was then protected with an Alloc group before a ring closure reaction to form the B ring and subsequent deprotection to give the product. The key stage in this synthesis is described as the ring closure to form the B ring which occurs after the linking of the two A rings with the diether chain.

Using this route, to synthesise a number of dimers having the same monomer groups but different bridging groups requires the synthesis of each compound from scratch, i.e. the synthesis route is not able to readily produce a diverse collection of PBD dimers, where the diversity is in the dimer bridge.

A second synthesis method for compounds of formula I is disclosed in co-pending application GB 0404577.9 (filed 1 Mar. 2004) incorporated herein by reference.

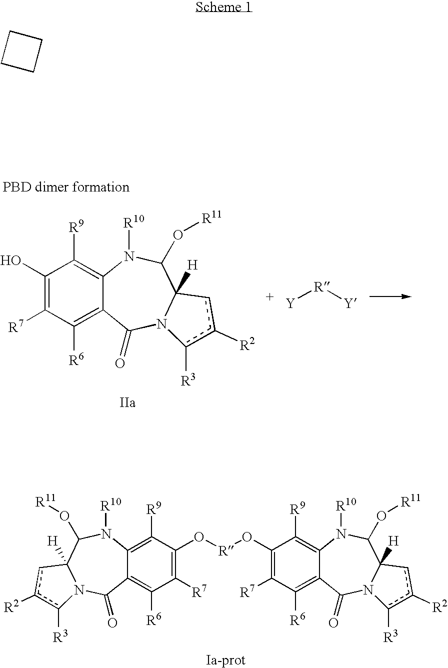

The PBD dimer compound I may be synthesized by dimerisation of PBD monomer compounds following deprotection of the OH group at the C8 position. The synthesis route illustrated in scheme 1 shows compounds where both PBD monomer groups have the same substituent pattern.

The protected dimer Ia-prot may be formed from PBD monomer compounds through reaction with a disubstituted linking chain. The linking chain is preferably of the general form Y—R″—Y′ where R″ is as previously defined and Y and Y′ are groups which can be reacted with an alcohol to form an ether linkage. Y and Y′ are preferably independently selected from I, Br, Cl, OH, mesylate or tosylate.

In a preferred aspect, Y and Y′ are the same. In a preferred aspect Y and Y′ are both iodo- groups.

Where Y and/or Y′ is I, Br, Cl, mesylate or tosylate, the Y—R″—Y′ reactant is coupled to the PBD monomer compound by a simple elimination reaction with Y and Y′ as leaving groups. For example where the linking chain is —O—CH₂—CH₂—CH₂—O—, the PBD monomer is reacted with 1,3-diiodopropane in the presence of K₂CO₃. Generally, where the linking chain is a straight chain alkyl ether of the form —O—(CH₂)ₙ—O—, the PBD monomer is preferably reacted with the corresponding 1,n-diiodoalkane.

Where Y and/or Y′ is OH, the Y—R″—Y′ reactant is coupled to the PBD monomer under Mitsunobu conditions.

It is important that the OH protecting group at C11 in the PBD monomer is orthogonal to the OH protecting group at C8. This allows the C8 protection to be removed to give the free alcohol to allow dimerisation whilst the C11 OH group remains protected and therefore unreactive under the dimerisation conditions.

Following dimerisation, the imine bond in the compound of formula Ia-prot can be deprotected by standard methods to yield the unprotected compound I (which may be in its carbinolamine or carbinolamine ether form , depending on the solvents used). For example if R¹⁰ is Alloc, then the deprotection is carried out using palladium to remove the N10 protecting group, followed by the elimination of water. If R¹⁰ is Troc, then the deprotection is carried out using a Cd/Pb couple to yield the compound of formula I.

If the nitrogen protecting group (R¹⁰) is such that the desired end product still contains it, e.g. if it is removable in vivo, then the C11 deprotected form of compound of formula I may be synthesised by removal of the oxygen protecting group under suitable conditions to leave the R¹⁰ group in unaffected.

The above described methods are suited to the synthesis of dimers where both the PBD monomers have the same substituent pattern. One method of synthesising a dimer where the substituent pattern of the two PBD monomers is not the same involves protecting one end of the compound Y—R″—Y′ (or using an already protected compound), coupling a PBD monomer to the unprotected end, deprotecting the other end and coupling a different PBD monomer to the free end. This route is shown in scheme 2.

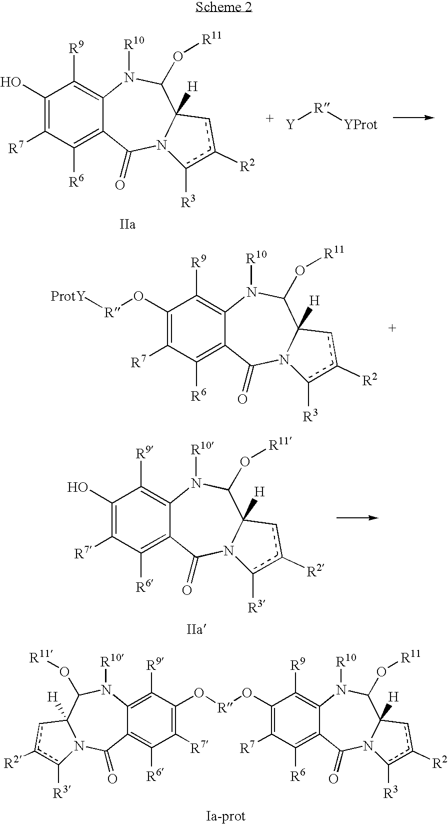

where Yprot, is a protected version, or precursor to Y′. If Y′ is protected then the protecting group used should be orthogonal to those on the rest of the molecule, in particular, R¹⁰ and R¹¹. One example of this route, would be to have Y as —OH and YProt as —0-benzyl. The first monomer could be joined by Mitsunobu coupling, the benzyl hydroxy deprotected, and then the free hydroxy coupled to the second monomer by a further Mitsunobu reaction.

### Further Preferences

The following preferences apply to formula I. The preferences may be combined together in any combination.

R⁹ is preferably H.

R² is preferably R, and is more preferably an optionally substituted C₅₋₂₀ aryl group or a C₁₋₇ alkyl group. Most preferred is a ═CH₂ group. R⁶ is preferably selected from H, OH, OR, SH, NH₂, nitro and halo, and is more preferably H or halo, and most preferably is H.

R⁷ is preferably independently selected from H, OR, SH, SR, NH₂, NHR, NHRR′, and halo, and more preferably independently selected from H and OR, where R is preferably selected from optionally substituted C₁₋₇ alkyl, C₃₋₁₀ heterocyclyl and C₅₋₁₀ aryl groups. Most preferably R⁷ is OCH₃.

R¹⁰ is preferably BOC or Troc. R¹¹ is preferably THP or a silyl oxygen protecting group (for example TBS). More preferably, R¹⁰ and R¹⁵ together form a double bond between N10 and C11.

R″ is preferably a C₃₋₁₂ alkylene group and each X is preferably O. More preferably, R″ is a C_{3 or 5} alkylene chain and each X is O, with a R″ being C₃ propylene in the most preferable embodiments.

It is further preferred that the substituent groups on all positions of each mononmer unit that make up the dimer are the same.

In preferred aspects of the present invention, the compounds of formula I are substituted as shown in formula III.

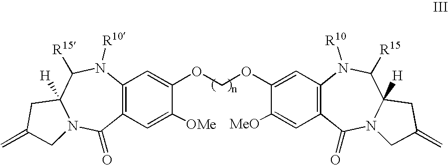

In compounds of formula III:

preferably n is 3 or 5;

R¹⁰ is preferably BOC or Troc; and R¹⁵ is preferably OH or OR¹¹ where R¹¹ is preferably THP or a silyl oxygen protecting group (for example TBS);

more preferably, R¹⁰ and R¹⁵ together form a double bond between N10 and C11.

In most preferred compounds of formula III, n is 3 or 5 and R¹⁰ and R¹⁵ together form a double bond between N10 and C11 i.e.

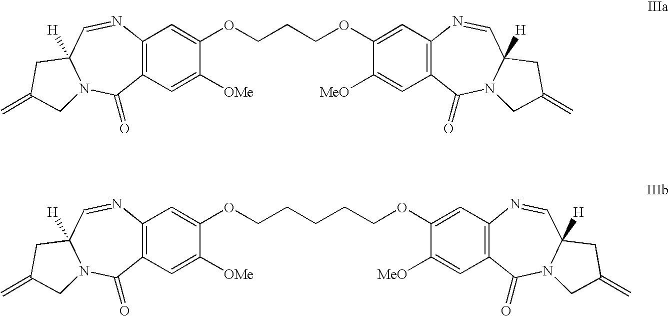

As discussed above, these compounds may be in a solvate form, for example with water or an alcohol, such as methanol, added across the imine bond:

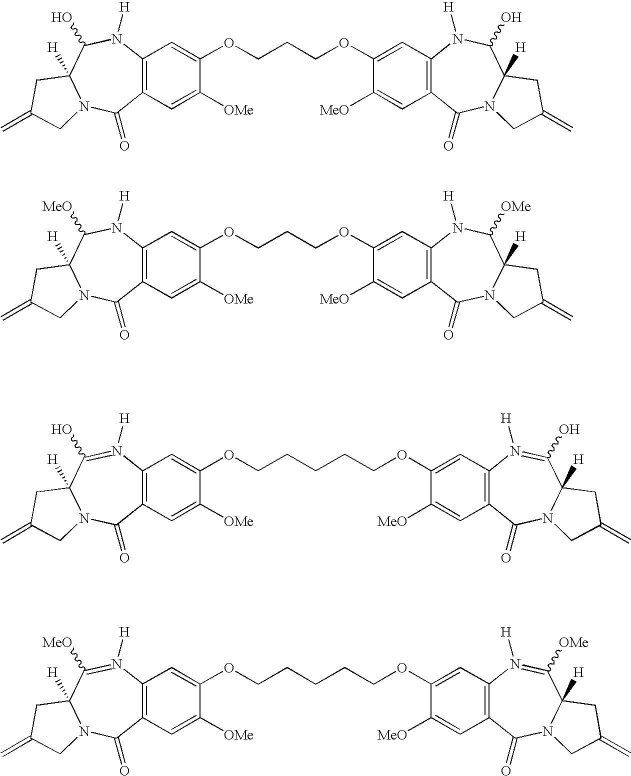

### EXAMPLES

### Example 1: Synthesis of the PBD Dimer SJG-136

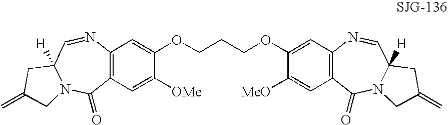

SJG-136 is a pyrrolobenzodiazepine (PBD) dimer according to formula I that is a sequence-selective DNA interstrand cross-linking agent. It comprises two PBD monomeric units ^{3,4} joined through their C8-positions via a propyldioxy linker, with each PBD C-ring containing a C2-exo-methylene functionality.^{5,6} The molecule has been shown to interact in the minor groove of DNA, spanning a total of six base pairs and alkylating the N2-positions of guanine bases situated on opposite strands of the DNA but separated by two base pairs. NMR, molecular modeling and gel electrophoresis-based studies on SJG-136 and related analogues suggest that it prefers to bind to Pu-GATC-Py sequences (Pu=purine; Py=pyrimidine), a feature that can be explained by hydrogen bonding interactions between the drug and certain molecular features of the DNA bases.⁷⁻⁹ The SJG-136 adduct provides a high degree of stabilisation towards melting of the duplex DNA as evidenced by energy calculations and an observed 33.6° C. increase in the thermal denaturation of calf thymus DNA after incubation for 18 hours at 37°P0 C. with SJG-136. ^{10,11} An NCI COMPARE analysis has shown that, although SJG-136 compares in general terms with DNA-binding agents, it does not fit within any of the clusters of known agents, including anthramycin and bizelesin.^{12,13}

Two methods of synthesising SJG-136 (shown above) are described in published PCT application WO/0012508.

### Example 2: Induction of Apoptosis in B-CLL Cells

### Patient Cells and Clinical Details

Peripheral blood samples from 34 patients with B-CLL (20 untreated and 14 treated) and 10 age-matched normal controls were obtained with the patients' informed consent. B-CLL was defined by clinical criteria as well as cellular morphology and the co-expression of CD19 and CD5 in lymphocytes simultaneously displaying restriction of light-chain rearrangement. Staging was based on the Binet classification system.¹⁴ None of the previously treated patients had received therapy for at least three months prior to this study. V_{H} gene mutational status was determined for all 34 patients using the method described previously.¹⁵ The resulting PCR products were sequenced and were considered unmutated if they showed homology of 98% or higher with the closest germ line sequence. The clinical characteristics of the patient cohort are summarized in Table 1.

TABLE 1

| | |
|---|---|
| No. of Cases | 34 |
| Mean age (years) | 67 |
| Sex (Male/Female) | 21/13 |
| Binet Stage (A/B/C) | 17/4/13 |
| Previous treatment | 20/14 |
| (Untreated/Treated) | |
| V_{H} mutation (Mutated/Unmutated) | 18/16 |

### Primary B-CLL Cell Culture Conditions

Freshly isolated peripheral blood lymphocytes (1×10⁶/ml) were cultured in Eagles medium (Invitrogen, Paisley, UK) supplemented with 100 units/ml penicillin, 100 μg/ml streptomycin and 10% fetal calf serum. Lymphocytes were incubated at 37° C. in a humidified 5% carbon dioxide atmosphere in the presence of SJG-136 (10⁻¹⁰−10⁻⁷ M). Parallel experiments using chlorambucil (10⁻⁶−5×10⁻⁵ M):

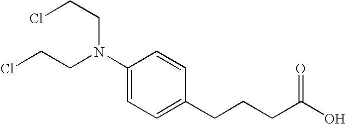

and fludarabine (10⁻⁷−10⁻⁵ M):

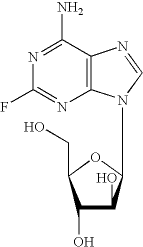

were also performed in order to assess the comparative intra- and inter-sample in vitro cytotoxicity. In addition, control cultures were carried out to which no drug was added to normal and leukemic lymphocytes. Cells were subsequently harvested by centrifugation and were analyzed by flow cytometry using the methods outlined below. Experiments were performed either in duplicate or triplicate.

### Cell Lines

The ability of SJG-136 to induce apoptotic cell death was investigated in the p53 non-expressing/mutant leukemic cell lines, K562 (chronic myelogenous leukemia) and MOLT-4 (T-cell acute lymphoblastic leukemia) containing a G>A mutation at codon 248 of the p53 gene. Cells were maintained in RPMI 1640 (Invitrogen) with 10% fetal calf serum, 100 units/ml penicillin, and 100 μg/ml streptomycin in a humidified atmosphere with 5% CO₂. Cells were cultured for 48 h in the presence or absence of SJG-136, chlorambucil or fludarabine at the concentrations given previously. Apoptosis was measured by Annexin V labeling (Dako, Ely, UK) and was quantified using flow cytometry.

### Measurement of In Vitro Apoptosis

In this study changes in forward light scatter (FSC) and side light scatter (SSC) characteristics were used to quantify apoptotic and viable cell populations as described previously.¹⁶⁻¹⁸ Typically, lymphocytes show a reduction in FSC (a function of cytoplasmic shrinkage) and an increase in SSC (due to increased granularity) when they undergo apoptosis.¹⁹ The quantitation of apoptosis using an FSC/SSC gating strategy in conjunction with back gating of R-phycoerythrin cyanine 5 (RPE-cy5) labeled CD19+ (Dako) or R-phycoerythrin labeled CD3+ (Dako) lymphocytes allowed simultaneous acquisition of data in viable and apoptotic B-lymphocyte and T-lymphocyte sub-populations, respectively. All LD₅₀ and LD₉₀ values (the concentration of the relevant chemotheraputic agent required to kill 50% and 90% of cells respectively) were derived from the dose-response curves. Duplicate samples were assessed using fluorescein (FITC)-labeled Annexin V to confirm the presence of apoptotic cells in the cell cultures and to validate the FSC/SSC quantitation method.²⁰

### SJG-136 Induced Caspase-3 Activation

B-CLL cells were incubated at 37° C. in a humidified 5% carbon dioxide atmosphere in the presence of SJG-136 (10⁻¹⁰−10⁻⁷ M) or fludarabine (10⁻⁷″10⁻⁵ M) for 12, 24 and 48 h. Cells were then harvested by centrifugation and labeled with CD19 RPE-cy5 conjugated antibody. Subsequently the cells were incubated for 1 h at 37° C. in the presence of the PhiPhiLux™ G₁D₂ substrate (Calbiochem, Nottingham, UK). The substrate contains two fluorophores separated by a quenching linker sequence that is cleaved by active caspase-3. Once cleaved the resulting products fluoresce green and can be quantified using flow cytometry. In additional experiments the caspase-8 inhibitor, Z-IETD-FMK, or the caspase-9 inhibitor, Z-LEHD-FMK, (Cambridge Bioscience, Cambridge, UK) were added to SJG-136-treated cell cultures (final concentration 2 μM) in order to determine whether either of these inhibitors was able to abrogate the apoptotic effects of SJG-136 in B-CLL cells. The activation of caspase-3 was partially abrogated by the addition of the caspase-9 inhibitor, Z-LEHD-FMK, but not by the caspase-8 inhibitor, Z-IETD.FMK, indicating that SJG-136-induced apoptosis is predominantly mediated through the intrinsic apoptotic pathway.

### Statistical analysis

The data obtained in these experiments were evaluated using the equal variance and paired Student's t-test, and correlation coefficients were calculated from least squares linear regression plots. LD₅₀ values were calculated from line of best-fit analysis of the dose response curves. All statistical analyses were performed using Graphpad Prism 3.0 software (Graphpad Software Inc., San Diego, Calif.).

### Measurement of Apoptosis in B-CLL Cells

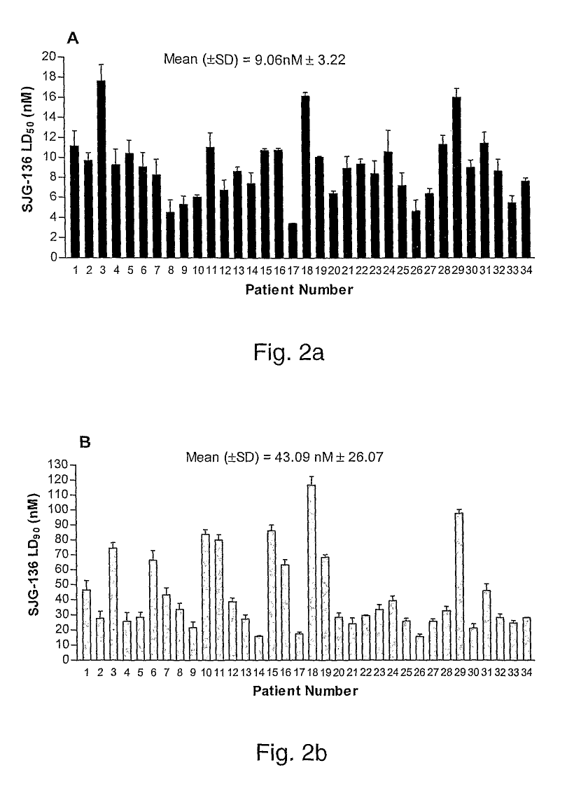

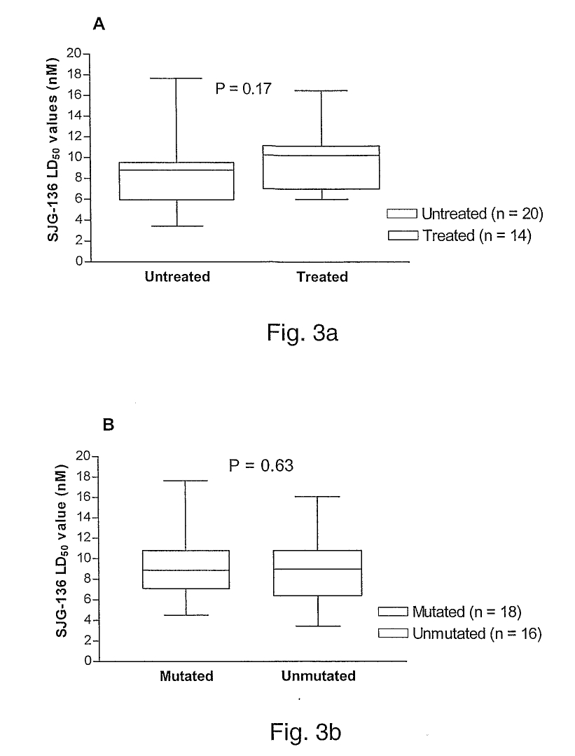

A flow cytometry-based in vitro apoptosis detection assay was used to determine whether SJG-136 could induce apoptotic cell death in B-CLL cells. The characteristic changes in the forward and side light scatter resulting from cellular shrinkage described previously were used to define apoptosis.¹⁹ In addition, Annexin V labeling was also performed in order to verify the light scatter data. Apoptosis was induced in all 34 patient samples following exposure to SJG-136 with a mean LD₅₀ value (±SD) of 9.06 nM (±3.2 nM) and a mean LD₉₀ value (±SD) of 43.09 nM (±26.1 nM) (FIG. 2a and 2b respectively). There was no significant difference in the LD₅₀ values between the treated and untreated patient groups (FIG. 3a).

Similarly, there was no significant difference in LD₅₀ values when the patient cohort was analyzed according to mutational status (FIG. 3b). Furthermore, two of the patients in the treated patient group had a known p53 mutation and showed a high degree of in vitro resistance to fludarabine but demonstrated similar sensitivity to SJG-136 when compared with the remaining patient samples.

### Example 3: SJG-136 Cytotoxicity in p53 Mutant B-CLL

Two of the patients in the B-CLL cohort described in example 2 had known p53 mutations that were associated with both clinical and in vitro resistance to a common chemotherapeutic agent, fludarabine. Both of these patients showed similar SJG-136 in vitro sensitivity to the rest of the patient cohort indicating that p53 activation was probably not required for effective SJG-136 cell killing.

Since SJG-136 is a DNA minor groove interstrand cross-linking agent, it was investigated whether SJG-136 induced the phosphorylation of p53 and stimulated downstream nucleotide excision repair in B-CLL cells as evidenced by the induction of GADD45.

GADD45 protein expression is up-regulated following p53 activation in response to DNA damage and is responsible for orchestrating nucleotide excision repair. The cellular responses of B-CLL cells to chlorambucil and SJG-136 were compared to determine whether these two cross-linking agents both induced phosphorylation of p53 and activated downstream nucleotide excision repair.

B-CLL cells were cultured for 4h and 48h in the presence or absence of one of the drugs under investigation. Cells were harvested by centrifugation and incubated with 10 μL of anti-CD19-RPE-cy5 conjugated antibody. Subsequently the cells were washed with phosphate buffered saline (PBS) at pH 7.2 and then prepared for intracellular staining of phosphorylated p53 and GADD45 (Santa Cruz Biotechnology, Santa Cruz, Calif.) using a commercially available kit (DAKO, Ely, UK). A FITC-labeled secondary antibody was added to the cells (DAKO) and after a final washing step the cells were resuspended in 0.5 mL of 1% paraformaldehyde prior to flow cytometric analysis using a FACScan flow cytometer (Becton Dickinson, Calif.).

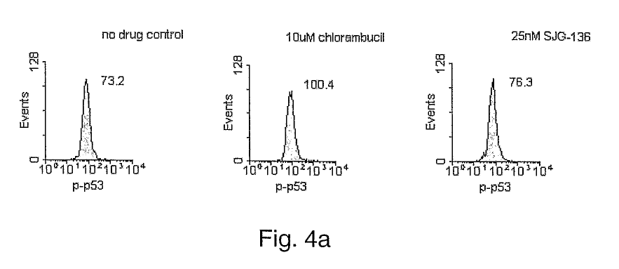

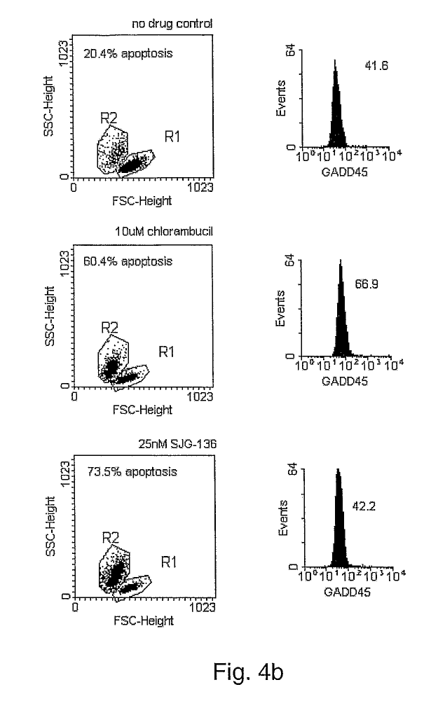

Phosphorylation of p53 and expression of GADD45 were measured in control B-CLL cell samples with no cytotoxic agent added and in B-CLL cell samples in the presence of either chloambucil or SJG-136. The results for p53 phosphorylation and GADD45 expression are shown in FIGS. 4a and 4b respectively.

FIG. 4a shows the increase in phosphorylated p53 (p-p53) in the presence of 10 μM chlorambucil when compared to both the control experiment with no chemotherapeutic agent present, and the experiment in the presence of 25 nM SJG-136.

FIG. 4b shows both the degree of apoptosis in the cell culture and the level of GADD45 expression for B-CLL cultures in the presence of chlorambucil, SJG-136, and in a control experiment with no chemotherapeutic agent present. The results show that in the presence of chlorambucil, whilst apoptosis is significantly increased over the control experiment, GADD45 expression is also much higher. This indicates that chlorambucil is acting on a p53 mediated apoptosis pathway. SJG-136 on the other hand, shows a greater degree of apoptosis than either the control experiment or the chlorambucil experiment. In addition, the level of GADD45 expression is only slightly higher than in the control experiment. This indicates that SJG-136 is acting primarily on an apoptosis pathway that is not regulated by p53.

From FIGS. 4a and 4b, it is clear that none of the B-CLL cells (n=9) treated with SJG-136 phosphorylated p53 or up-regulated GADD45 expression to any significant degree following exposure to SJG-136. In contrast, when the same patient samples were treated with the cross-linking agent chlorambucil they showed an increase in phosphorylated p53 and a marked increase in GADD45 expression over the same culture period.

### Example 4

### Comparison of the Cytotoxic Effects of SJG-136 and Fludarabine in Treated and Untreated Patients

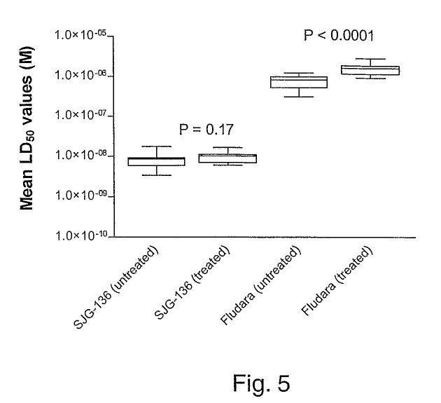

Preparation of cells and measurement of apoptosis was conducted as in example 2. B-CLL cells were cultured in the presence of fludarabine and separately in the presence of SJG-136. The mean LD₅₀ values for cells taken from both previously treated and previously untreated patients is shown in FIG. 5.

The previously treated patient group had undergone at least one previous treatment for B-CLL with a known therapeutic compound. Known therapeutic compounds used in this study were chlorambucil, fludarabine (both with and without cyclophosphamide) or CHOP (cyclophosphamide, doxorubicin, vincristine and prednisolone). These treatments with known therapeutic compounds were undertaken in line with known therapeutic procedures.

In FIG. 5, the previously treated patient sub-set demonstrated a significantly higher mean LD₅₀ value for fludarabine when compared with the untreated sub-set (P<0.0001). In contrast, previous treatment appeared to have no effect on the cytotoxicity of SJG-136 (P=0.17) in cells taken from the same patient cohort.

### Example 5

### Differential Cytotoxicity of SJG-136 in B- and T-cells from Normal and from B-CLL Patients

B- and T-lymphocytes from 10 healthy normal control patients were assessed for their sensitivity to SJG-136-induced apoptosis. In addition, the T-lymphocytes from 12 B-CLL patients from the untreated patient group, described in example 2, whose T-lymphocyte population was greater than 5% of the total lymphocyte population were also analyzed in order to determine whether SJG-136 had differential cytotoxic effects on the various lymphocyte sub-populations. None of the treated patient samples met this criterion and were therefore not analyzed.

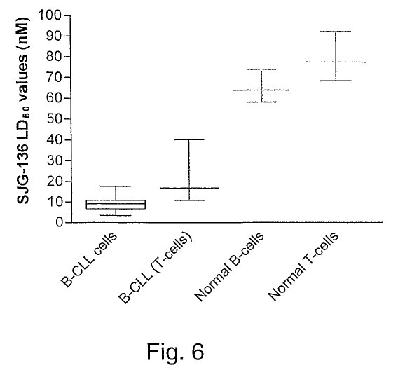

The T-cells from the B-CLL samples showed consistently higher LD₅₀ values than their corresponding malignant B-cell clones (P=0.0006; paired t-test). In addition, the healthy normal control B—and T-lymphocytes demonstrated higher LD₅₀ values than the B-CLL cells (P<0.0001 and P<0.0001 respectively). The relative sensitivities of the various lymphocyte populations to SJG-136 are illustrated in FIG. 6.

### Comparative Example 1

### Differential Cytotoxicity of Fludarabine and Chloambucil in B- and T-cells from Normal and from B-CLL Patients

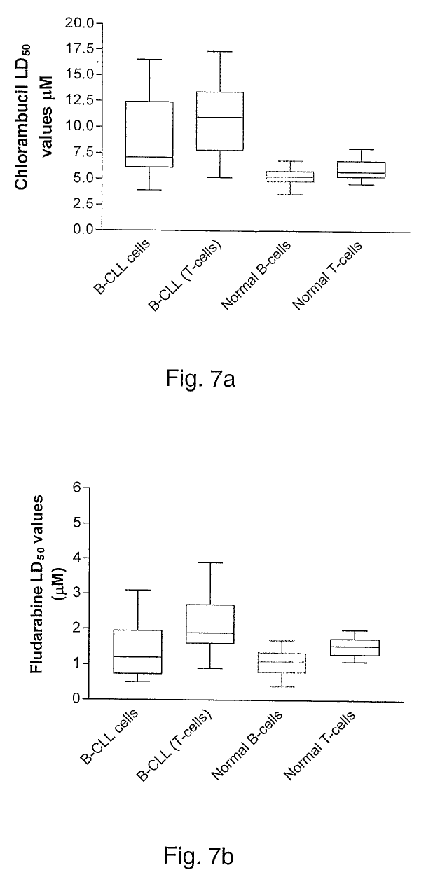

The sensitivity of normal B- and T- cells and B- and T- cells from B-CLL patients were assessed for their sensitivity to current chemotherapeutic agents fludarabine and chlorambucil using the same methods as in example 5. The LD₅₀ values for chlorambucil and fludarabine are shown in FIGS. 7a and 7b respectively.

From FIGS. 7a and 7b, it is clear that the mean LD₅₀ values for both chlorambucil and fludarabine are lower in normal B- and T- cells than in corresponding B- and T- cells from B-CLL patients i.e. normal cells are killed more easily by these agents than are B-CLL cells.

Comparison of FIGS. 7a and 7b with FIG. 6 clearly shows the differential killing of malignant cells over normal cells by SJG-136.

### Example 6

### Induction of Apoptosis in B-CLL Cells

The investigation of apoptosis in B-CLL cells, described in example 2, was extended by inclusion of a further 12 patients to give an expanded patient cohort of 46. The experimental methods and analysis were performed as described in example 2. The clinical characteristics of the expanded patient cohort are summarised in table 2.

TABLE 2

| | |
|---|---|
| No. of Cases | 46 |
| Mean age (years) | 65 |
| Sex (Male/Female) | 30/16 |
| Binet Stage (A/B/C) | 23/8/15 |
| Previous treatment | 26/20 |
| (Untreated/Treated) | |
| V_{H} mutation (Mutated/Unmutated) | 25/21 |

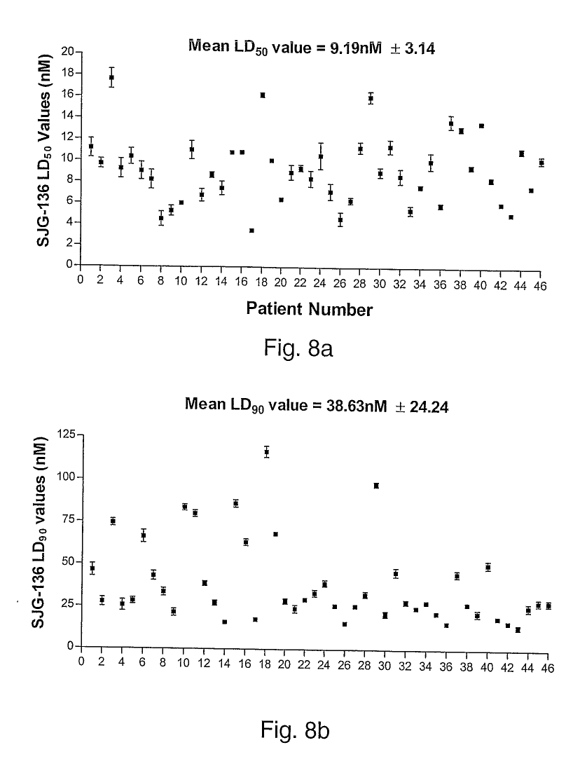

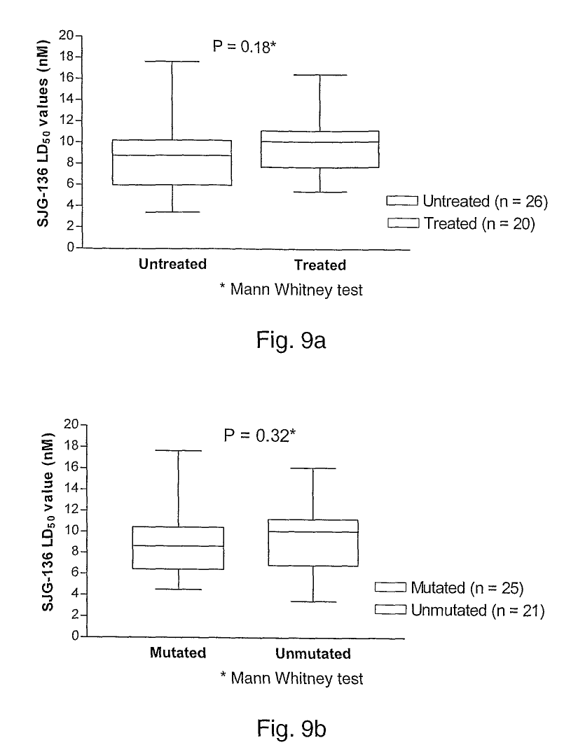

The results obtained from the expanded patient cohort were found to be entirely consistent with those presented in example 2. Apoptosis was induced in all 46 patient samples following exposure to SJG-136 with a mean LD₅₀ value (±SD) of 0.19 nM (±3.14 nM) and a mean LD₉₀ value (±SD) of 38.63 nM (±24.24 nM) (FIG. 8a and 8b respectively). There was no significant difference in the LD₅₀ values between the treated and untreated patient groups (FIG. 9a). Similarly, there was no significant difference in LD₅₀ values when the patient cohort was analyzed according to mutational status (FIG. 9b).

### Example 7

### Comparison of the Cytotoxic Effects of SJG-136 and Fludarabine in Treated and Untreated Patients

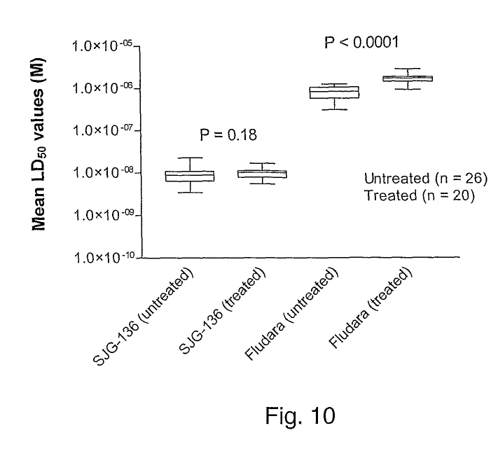

The investigation of cytoxicity in B-CLL cells, described in example 4, was expanded by using the expanded patient cohort described in example 6, i.e. the same additional 12 patients. The experimental methods and analysis were performed as described in example 4. The results obtained from the expanded patient cohort of 46 patients were found to be entirely consistent with those presented in example 4. The mean LD₅₀ values for cells taken from both previously treated and previously untreated patients in the expanded patient cohort are shown in FIG. 10.

Consistent with example 4, in FIG. 10 the previously treated patient sub-set demonstrated a significantly higher mean LD₅₀ value for fludarabine when compared with the untreated sub-set (P<0.0001). Also consistent with example 4, the results with fludarabine contrasted with those of SJG-136, in that previous treatment status appeared to have no effect on the cytotoxicity of SJG-136 (P=0.18).

### REFERENCES (which are incorporated herein by reference)

1. Ries, L. A. G., Miller, B. A., Hankey, B. F., Eisner, M. P., Mariotto, A., Fay, M. P., Feuer, E. J., and Edwards, B. K. SEER cancer statistics review, 1972-1991: tables and graphs., pp 94-2789, National Cancer Institute Publication, NIH Bethesda, Md., 1994.

2. Lee, J. S., Dixon, D. O., Kantarjian, H. M., Keating, M. J., and Talpaz, M. Prognosis in chronic lymphocytic leukemia: a multivariate regression analysis of 325 untreated patients. Blood. 69: 929-936, 1987.

3. Thurston, D. E. Advances in the Study of Pyrrolo[2,1-c][1,4]benzodiazepine (PBD) Antitumour Antibiotics. Molecular Aspects of Anticancer Drug-DNA Interactions; pp 54-88, The Macmillan Press Ltd., London, UK: London, 1993.

4. Thurston, D. E. Nucleic Acid Targeting: Therapeutic Strategies for the 21st Century. Br J Cancer, 80: 65-85, 1999.

5. Gregson, S. J., Howard, P. W., Hartley, J. A., Brooks, N. A., Adams, L. J., Jenkins, T. C., Kelland, L. R., Thurston, D. E. Design, synthesis, and evaluation of a novel pyrrolobenzodiazepine DNA-interactive agent with highly efficient cross-linking ability and potent cytotoxicity. J Med Chem., 44: 737-748, 2001.

6. Gregson, S. J., Howard, P. W., Jenkins, T. C., Kelland, L. R., and Thurston, D. E. Synthesis of a novel C2/C2 ′-Exo unsaturated pyrrolobenzodiazepine cross-linking agent with remarkable DNA binding affinity and cytotoxicity. J Chem Soc Chem Commun., 797-798, 1999.

7. Mountzouris, J. A., Wang, J. J., Thurston, D., and Hurley, L. H. Comparison of a DSB-120 DNA Interstrand Cross-Linked Adduct with the Corresponding Bis-Tomaymycin Adduct—an Example of a Successful Template-Directed Approach to Drug Design Based Upon the Monoalkylating Compound Tomaymycin. J Med Chem., 37: 3132-3140, 1994.

8. Jenkins, T. C., Hurley, L. H., Neidle, S., and Thurston, D. E. Structure of a Covalent DNA Minor-Groove Adduct With a Pyrrolobenzodiazepine Dimer—Evidence For Sequence-Specific Interstrand Cross-Linking. J Med Chem., 37: 4529-4537, 1994.

9. Smellie, M., Bose, D. S., Thompson, A. S., Jenkins, T. C., Hartley, J. A., and Thurston, D. E. Sequence-Selective Recognition of Duplex DNA Through Covalent Interstrand Cross-Linking: Kinetic and Molecular Modeling Studies With Pyrrolobenzodiazepine Dimers. Biochem., 42: 8232-8239, 2003.

10. Adams, L. J.; Jenkins, T. C.; Banting, L.; Thurston, D. E. Molecular Modelling of a Sequence-Specific DNA-Binding Agent Based on the Pyrrolo [2,1-c] [1,4]benzodiazepines. Pharmacy & Pharmacology Communications 1999, 5, 555-560.

11. Gregson, S. J., Howard, P. W., Gullick, D. R., Hamaguchi, A., Corcoran, K. E. et al. Linker Length Modulates DNA Cross-Linking Reactivity and Potency for Ether-Linked C2-Exo-Unsaturated Pyrrolo [2,1-c] [1,4]benzodiazepine (PBD) Dimers. J Med Chem., (In Press) 2003.

12. Alley, M. C., Hollingshead, M. G., Pacula-Cox, C. M., Waud, W. R., Hartley, J. A., Howard, P. W., Gregson, S. J., Thurston, D. E., and Sausville, E. A. SJG-136 (NSC 694501) A Novel Rationally Designed DNA Minor Groove Interstrand Cross-Linking Agent With Potent and Broad Spectrum Antitumour Activity. Part 2: Efficacy Evaluations. Cancer Res., (In Revision) 2003.

13. Hartley, J. A., Spanswick, V. J., Brooks, N., Clingen, P. H., McHugh, P. J., Hochhauser, D., Pedley, R. B., Kelland, L. R., Alley, M. C., Schultz, R., Hollingshead, M. G., Sausville, E. A., Gregson, S. J., Howard, P. W., and Thurston, D. E. SJG-136 (NSC 694501) A Novel Rationally Designed DNA Minor Groove Interstrand Cross-Linking Agent With Potent and Broad Spectrum Antitumour Activity. Part 1: Cellular Pharmacology, In Vitro and Initial In Vivo Antitumor Activity. Cancer Res., (In Revision) 2003.

14. Binet, J. L., Auquier, A., Dighiero, G., Chastang, C., Piguet, H., Goasguen, J., Vaugier, G., Potron, G., Colona, P., Oberling, F., Thomas, M., Tchernia, G., Jacquillat, C., and Boivin, P. A new prognostic classification of chronic lymphocytic leukemia derived from a multivariate survival analysis. Cancer, 48: 198-206, 1981.

15. Starczynski, J., Pepper, C., Pratt, G., Hooper, L., Thomas, A., Hoy, T., Milligan, D., Bentley, P., and Fegan, C. The P2X7 receptor gene polymorphism 1513A→C has no effect on clinical prognostic markers, in vitro sensitivity to fludarabine, Bc1-2 family protein expression or survival in B-cell chronic lymphocytic leukaemia. Br J Haematol., 122: 66-71, 2003.

16. Pepper, C., Thomas, A., Hoy, T., and Bentley, P. Chlorambucil resistance in B-cell chronic lymphocytic leukemia is mediated through failed Bax induction and selection of high Bc1-2-expressing subclones. Br J Haematol., 104: 581-588, 1999.

17. Pepper, C., Hooper, K., Thomas, A., Hoy, T., and Bentley, P.

Bc1-2 antisense oligonucleotides enhance the cytotoxicity of Chlorambucil in B-cell chronic lymphocytic leukemia cells. Leuk Lymphoma. 42: 491-498, 2001.

18. Pepper C, Thomas A, Hoy T. et al. The vitamin D3 analog, EB1089, induces apoptosis via a p53-independent mechanism involving p38 MAP kinase activation and suppression of ERK activity in B-cell chronic lymphocytic leukemia cells in vitro. Blood. 101: 2454-2460, 2003.

19. Ferlini, C., Di Cesare, S., Rainaldi, G., Malorni, W., Samoggia, P., Biselli, R., and Fattorossi, A. Flow cytometric analysis of the early phases of apoptosis by cellular and nuclear techniques. Cytometry, 24: 106-115, 1996.

20. Vermes, I., Haanen, C., Steffens-Nakken, H., and Reutelingsperger, C. A novel assay for apoptosis: flow cytometric detection of phosphatidylserine expression on early apoptotic cells using fluorescein labeled Annexin V. J Immunol Methods. 184: 39-51, 1995.

1-10. (canceled)

11. A method of treatment of a patient suffering from leukaemia that exhibits drug resistance, comprising administering to said patient a therapeutically effective amount of a compound of formula I: 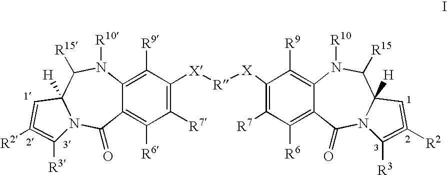
or a pharmaceutically acceptable salt or solvate thereof, wherein:
the dotted lines indicate the optional presence of a double bond between C1 and C2 or C2 and C3;
R² and R³ are independently selected from —H, ═O, ═CH₂, —CN, —R, OR, halo, ═CH—R, O—SO₂p13 R, CO₂R and COR;
R⁶, R⁷ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R¹⁰ is a carbamate-based nitrogen protecting group and R¹⁵ is either O—R¹¹, wherein R¹¹ is an oxygen protecting group, or OH, or R¹⁰ and R¹⁵ together form a double bond between N10 and C11;
R″ is alkylene group, which chain may be interrupted by one or more heteroatoms and/or aromatic rings, and each X is independently selected from 0, S, or NH;
R^{2′}, R^{3′}, R^{6′}, R^{7′}, R^{9′}, R^{10′} and R^{15′} are all independently selected from the same lists as previously defined for R², R³, R⁶, R⁷, R⁹, R¹⁰ and R¹⁵ respectively.

12. The method according to claim 11, wherein the leukaemia comprises a p53 mutation.

13. The method according to claim 11 or claim 12, wherein the drug resistance of the leukaemia is a result of prior administration of a chemotherapeutic agent.

14. A method of treatment of a patient suffering from B-cell leukaemia wherein it is desired not to reduce the patient's T-cell count, comprising administering to said patient a therapeutically effective amount of a compound of formula I: 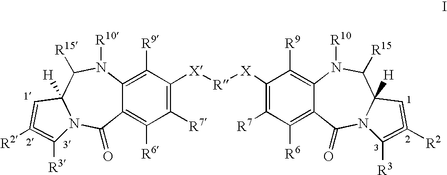
or a pharmaceutically acceptable salt or solvate thereof, wherein:
the dotted lines indicate the optional presence of a double bond between C1 and C2 or C2 and C3;
R² and R³ are independently selected from —H, ═O, ═CH₂, —CN, —R, OR, halo, ═CH—R, O—-SO₂R, CO₂R and COR;
R⁶, R⁷ and R⁹ are independently selected from H, R, OH, OR, SH, SR, NH₂, NHR, NRR′, nitro, Me₃Sn and halo;
where R and R′ are independently selected from optionally substituted C₁₋₁₂ alky, , C₃₋₂₀ heterocyclyl and C₅₋₂₀ aryl groups;
R¹⁰ is a carbamate-based nitrogen protecting group and R¹⁵ is either O—R¹¹, wherein R¹¹ is an oxygen protecting group, or OH, or R¹⁰ and R¹⁵ together form a double bond between N10 and c11;
R″ is a C₃₋₁₂ alkylene group, which chain may be interrupted by one or more heteroatoms and/or aromatic rings, and each X is independently selected from O, S, or NH;
R^{2′}, R^{3′}, R^{6′}, R^{7′}, R^{9′}, R^{10′} and R^{15′} are all independently selected from the same lists as previously defined for R², R³, R⁶, R⁷, R⁹, R¹⁰ and R¹⁵ respectively.

15. The method according to claim 14, wherein the compound exhibits higher cytotoxicity for B-cells than for T-cells in cells from healthy patients and in cells from those suffering from B-cell chronic lymphocytic leukaemia.

16. A method of treatment of a patient suffering from B-cell leukaemia wherein it is desired to selectively kill malignant B-cells, comprising administering to said patient a therapeutically effective amount of a compound of formula I: 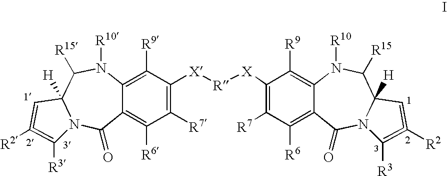
or a pharmaceutically acceptable salt or solvate thereof, wherein:
the dotted lines indicate the optional presence of a double bond between C1 and C2 or C2 and C3;
R² and R³ are independently selected from —H, ═O, ═CH₂, —CN, —R, OR, halo, ═CH—R, O—SO₂—R, CO₂R and COR;
R⁶, R⁷ and R⁹ are independently selected from H, R, OH, OR, SH, SR, NH₂NHR, NRR′, nitro, Me₃Sn and halo;
where R and R′ are independently selected from optionally substituted C₁₋₁₂, alkyl C₃₋₂₀ heterocyclyl and C₅₋₂₀ aryl groups;
R¹⁰ is a carbamate-based nitrogen protecting group and R¹⁵ is either O—R¹¹, wherein R¹¹ is an oxygen protecting group, or OH, or R¹⁰ and R¹⁵ together form a double bond between N10 and C11;
R″ is a C₃₋₁₂ alkylene group, which chain may be interrupted by one or more heteroatoms and/or aromatic rings, and each X is independently selected from O, S, or NH;
R^{2′}, R^{3′}, R^{6′}, R^{7′}, R^{9′}, R^{10′} and R^{15′} are all independently selected from the same lists as previously defined for R², R³, R⁶, R⁷, R⁹, R¹⁰ and R¹⁵ respectively.

17. The method according to claim 16, wherein the compound or pharmaceutically acceptable salt or solvate thereof shows a higher cytotoxicity towards malignant B-cell chronic lymphocytic leukaemia cells than towards normal B-cells.

18. The method according to either claim 11, 14 or 16, wherein the compound has the formula: 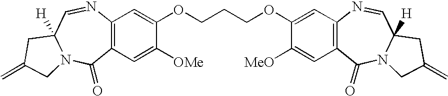
or is a pharmaceutically acceptable salt or solvate thereof.

19. The method according to claim 11, 14 or 16, wherein the leukaemia is B-cell chronic lymphocytic leukaemia.
